(19) United States
(12) Patent Application Publication
(10) Pub. No.: US 2022/0369733 A1
(43) Pub. Date: Nov. 24, 2022

(54) COVID-19 EXPOSURE DETECTOR

(71) Applicant: Terry Zore, Indianapolis, IN (US)

(72) Inventor: Terry Zore, Indianapolis, IN (US)

(21) Appl. No.: 17/324,597

(22) Filed: May 19, 2021

(51) Int. Cl. A41D 13/11; G01N 21/78; G16H 50/80; G01N 33/52

(52) U.S. Cl. CPC A41D 13/11; G01N 21/78; G16H 50/80; G01N 33/52; A41D 2300/20; A41D 2400/00

(57) ABSTRACT

The disclosure is the only product of its kind with the potential to curb the spread of contagious aerosolized disease via widely accessible and versatile disc that complements the requirement for wearing a face mask. CODIS is uniquely designed to enhance the contact tracing process of infectious diseases and serves as a proactive medium for identifying disease exposure in real-time. The disclosed pathogen indicator device includes a reagent configured to trigger a chemical reaction based on an exposure to a predetermined pathogen. The disclosure also includes a mask substrate configured to receive the reagent on a front side and to bond therewith on a backside thereof. The disclosure further includes a seal for the substrate and the reagent, the seal configured to provide a prophylactic temporary protection from the predetermined pathogen.

### BACKGROUND

COVID-19 is an infectious airborne disease that spreads through respiratory droplets. This disease has led to a global pandemic and claimed the lives of over a million people worldwide, yet, there lacks an ability to know in real-time when and if a person has been exposed to the virus. There have been no products available as original equipment or as an aftermarket to address this problem.

A device or apparatus to simplify the ability to detect in real-time if they have been exposed to the COVID-19 virus and other contagious diseases. There have been no products available as original equipment or as an aftermarket to address this problem either.

There exists a need for an apparatus or device such as the COVID-19 exposure detector, that is not being met by any known or disclosed device or system of present.

### SUMMARY OF THE INVENTION

The primary purpose of the COVID exposure detector is to serve as an efficient means of identifying COVID-19 and other contagious aerosolized diseases. The present invention overcomes the above referred to difficulties in helping individuals know in real-time when and if a person has been exposed to the virus. Thus preventing further spreading of the disease and helping to save lives. This invention does so in a simple and efficient manner that is easy to use.

The disclosure also includes a mask substrate configured to receive the reagent on a front side and to bond therewith on a backside thereof. The disclosure further includes a seal for the substrate and the reagent, the seal configured to provide a prophylactic temporary protection from the predetermined pathogen.

### BRIEF DESCRIPTION OF THE DRAWINGS

FIG. 1 is a front elevational view of the COVID-19 Exposure Detector device showing: Peel and stick COVID-19 exposure detector referenced as A., in accordance with an embodiment of the present disclosure.

FIG. 2 is a back elevational view of the COVID-19 Exposure Detector device showing: Adhesive backing referenced as B., in accordance with an embodiment of the present disclosure.

FIG. 3 is a side elevational view of the COVID-19 Exposure Detector device showing: Peel and stick COVID-19 exposure detector (changes color when exposed to COVID-19) referenced as A., and Adhesive backing referenced as B., in accordance with an embodiment of the present disclosure.

FIG. 4 is a top elevational view of the COVID-19 Exposure Detector device showing: Tear off, peel off packaging referenced as D., in accordance with an embodiment of the present disclosure.

FIG. 5 is a front perspective view of the COVID-19 Exposure Detector device showing: Dime size disc to be placed on mask between the nose and mouth referenced as C., in accordance with an embodiment of the present disclosure.

FIG. 6 is a side perspective view of the COVID-19 Exposure Detector device showing: Dime size disc to be placed on mask between the nose and mouth referenced as C., in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

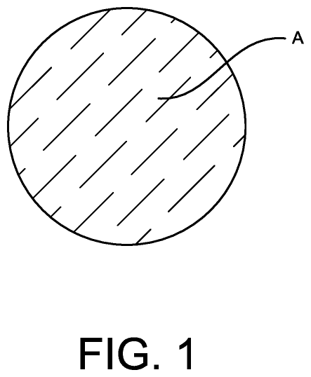

FIG. 1 is a front elevational view of the COVID-19 Exposure Detector device showing: Peel and stick COVID-19 exposure detector (changes color when exposed to COVID-19) referenced as A., in accordance with an embodiment of the present disclosure.

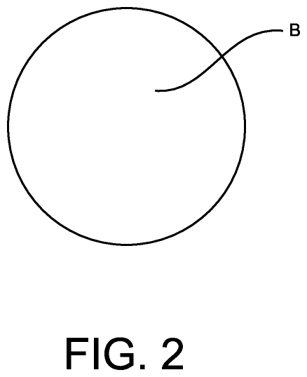

FIG. 2 is a back elevational view of the COVID-19 Exposure Detector device showing: Adhesive backing referenced as B., in accordance with an embodiment of the present disclosure.

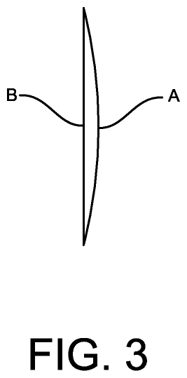

FIG. 3 is a side elevational view of the COVID-19 Exposure Detector device showing: Peel and stick COVID-19 exposure detector (changes color when exposed to COVID-19) referenced as A., and Adhesive backing referenced as B., in accordance with an embodiment of the present disclosure.

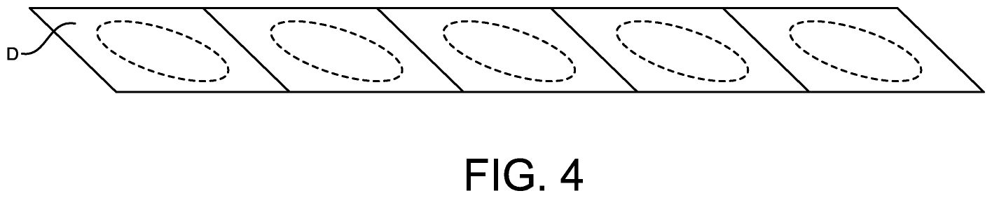

FIG. 4 is a top elevational view of the COVID-19 Exposure Detector device showing: Tear off, peel off packaging referenced as D., in accordance with an embodiment of the present disclosure.

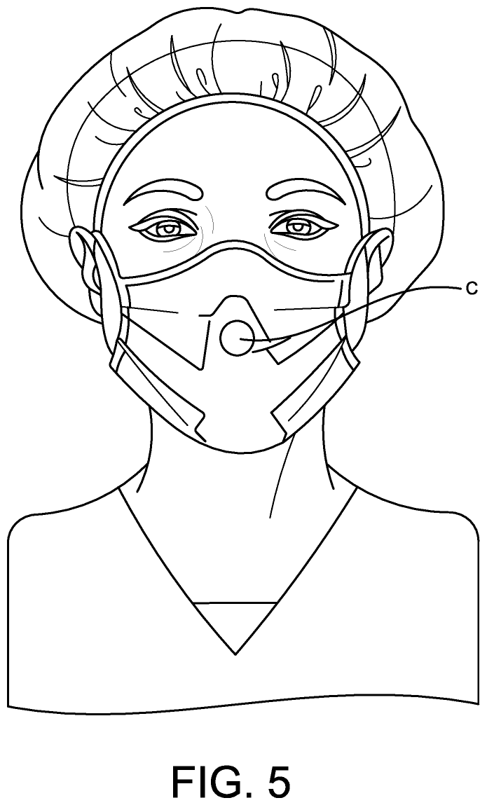

FIG. 5 is a front perspective view of the COVID-19 Exposure Detector device showing: Dime size disc to be placed on mask between the nose and mouth referenced as C., in accordance with an embodiment of the present disclosure.

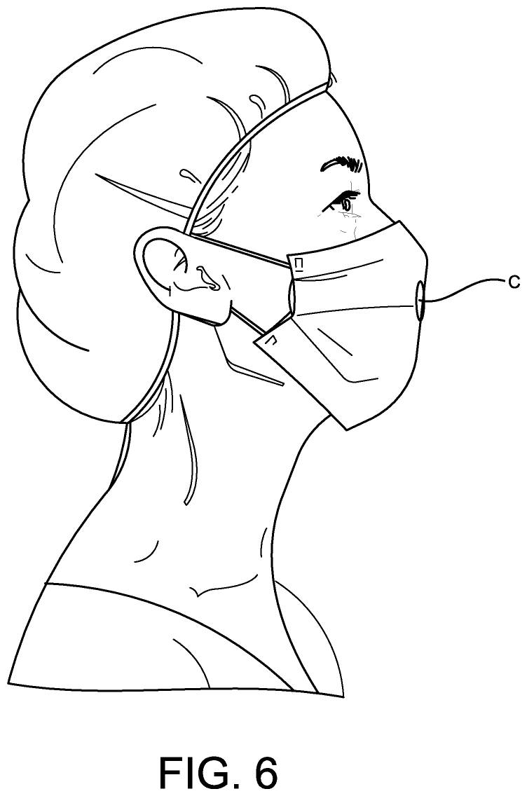

FIG. 6 is a side perspective view of the COVID-19 Exposure Detector device showing: Dime size disc to be placed on mask between the nose and mouth referenced as C., in accordance with an embodiment of the present disclosure.

The present disclosed COM exposure detector, also known as “CODIS”, is a dime-sized disc inserted with a COVID-19 exposure detector that can be placed on masks worn by individuals. Expanding upon the initial concept of pathogen detecting apparatuses, CODIS is placed on the front center of a face mask via the adhesive material on the hack of the product. Once placed on the mask, CODIS is able to alert the user within 5 to 30 minutes if the user has been exposed to a disease by a change in the detector's surface color. The CODIS disc contains a reagent that: ignites a chemical reaction if the user is exposed to a particular disease thus informing them within a relative time for them to take proper precautions, contact medical support and/or have a better recollection of who they have been in contact with and how long ago, all a means of identifying, isolating and combating virus exposure. To ensure the accuracy of detection, CODIS is packaged in a sealed wrap to prevent external air from contaminating the disc. Furthermore, CODIS can be utilized in various oxygen and temperature settings, granting it the ability to have a global impact. CODIS may prove to be a life-saving device with the potential to cease the spread of various infectious respiratory droplets.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

What is claimed is:

1. A pathogen indicator comprising:
a reagent that triggers a chemical reaction if a user thereof is exposed to a predetermined pathogen;
a substrate configured to receive the reagent on a front side and to attach to a surface on a backside thereof; and
a seal for the substrate and the reagent, the seal configured to provide a prophylactic temporary protection from the predetermined pathogen.

2. The pathogen indicator of claim 1, further comprising an adhesive backing on the substrate backside for an adherence to a face mask surface.

3. The pathogen indicator of claim 1, further comprising a plurality of pathogen indicators on an non-adhesive strip surface.

4. The pathogen indicator of claim 1, wherein the predetermined pathogen is Covid-19.

5. The pathogen indicator of claim 1, wherein the chemical reaction triggers a visual change in the reagent.

6. The pathogen indicator of claim 1, wherein the surface is an article of clothing of the user.

7. The pathogen indicator of claim 1, wherein the front side of the substrate is a convex surface of a button like object.

8. The pathogen indicator of claim 1, wherein a size of the substrate is similar to a size of a dime coin.

9. The pathogen indicator of claim 1, wherein the surface is a surgical face mask.

10. The pathogen indicator of claim 1, further comprising a breach of the prophylactic based on a breaking of the seal.

11. The pathogen indicator of claim 1, further comprising a predetermined time interval for the chemical reaction based on a concentration of the reagent.

12. The pathogen indicator of claim 1, wherein the chemical reaction is a change in a color of the reagent.

13. The pathogen indicator of claim 1, wherein a shape of the substrate is disc-like.

14. The pathogen indicator of claim 1, further comprising a placement of the pathogen indicator on a mask of the user between a corresponding mouth and a nose portion of the mask.

15. The pathogen indicator of claim 1, wherein the seal is a hermetic seal.

16. A pathogen indicator device comprising:
a reagent configured to trigger a chemical reaction based on an exposure to a predetermined pathogen;
a mask substrate configured to receive the reagent on a front side and to bond therewith on a backside thereof; and
a seal for the substrate and the reagent, the seal configured to provide a prophylactic temporary protection from the predetermined pathogen.

17. The pathogen indicator device of claim 16, further comprising a breach of the prophylactic based on a breaking of the seal.

18. The pathogen indicator of claim 16, further comprising a predetermined time interval for the chemical reaction based on a concentration of the reagent.

19. The pathogen indicator of claim 16, wherein the chemical reaction is a change in a color of the reagent.

20. The pathogen indicator of claim 16, further comprising a front side surface of the substrate configured to receive the predetermined pathogen via an aerosol transmission thereof.
